(19) United States
(12) Patent Application Publication
(10) Pub. No.: US 2013/0158635 A1
(43) Pub. Date: Jun. 20, 2013

(54) PAIN MANAGEMENT SYSTEM

(71) Applicants: Charles Federico, Cornelius, NC (US); Matthew Federico, Charlotte, NC (US); Gregory Federico, Cary, NC (US)

(72) Inventors: Charles Federico, Cornelius, NC (US); Matthew Federico, Charlotte, NC (US); Gregory Federico, Cary, NC (US)

(21) Appl. No.: 13/326,953

(22) Filed: Dec. 15, 2011

(51) Int. Cl. A61F 7/10

(52) U.S. Cl. 607/108; 607/112

(57) ABSTRACT

A pain management system that includes a support undergarment with a pouch configured to removeably secure a therapy pack, wherein the support undergarment comprises an anti-microbial material and at least one moisture barrier. The system further includes a therapy pack configured to form into a contoured design.

### BACKGROUND

Following surgical procedures, tissue swelling can cause pain and inflammation and inhibit healing of the affected area. Such tissue swelling can also be caused by trauma to the affected area. Treatment can include cold therapy such as a cold compress. Depending on the location of the affected area, application of a cold compress can limit the mobility of the subject of treatment. Treatment can further include compression of the affected area.

For example, after a vasectomy, a cold compress can be applied to a patient's groin region to reduce tissue swelling. Vasectomies have become a common outpatient medical procedure performed at a rate of over 500,000 per year in the United States. Traditional post-operative treatment protocol includes the intermittent use of cold therapy for a period of approximately one week after surgery. Cold therapy can be an uncomfortable process for patients. During treatment, a patient's mobility is generally restricted in order to maintain the position of a cold compress on the affected area.

Additionally, because the affected area contains broken skin, a patient is typically advised to minimize moisture at the incision site. Traditional cold therapy options create condensation on and near the affected area, which can increase the risk of infection and wound maceration. Achieving proper moisture balance around the wound site is important for effective healing.

Despite the prevalence of vasectomy procedures, there has long been a need for a system of managing pain while minimizing mobility restriction and the chance for infection.

### SUMMARY

In accordance with one embodiment, a pain management system can comprise a support undergarment with a pouch configured to removeably secure a therapy pack, wherein the support undergarment is at least partially formed with anti-microbial material and at least one moisture barrier. The system can further include a cold therapy pack, wherein the therapy pack is configured to form into a contoured design.

In one embodiment, the support undergarment can have support panels that are configured to provide stability to male genitals. In another embodiment, support undergarment is boxer-brief style underwear. In yet another embodiment, the support undergarment has moisture wicking material.

In one embodiment, the therapy pack comprises cold therapy gel. In another embodiment, the therapy pack comprises therapy beads. In yet another embodiment, the therapy pack comprises material configured to minimize the risk of frostbite when applied to male genitals.

### BRIEF DESCRIPTION OF THE DRAWINGS

FIG. 1 is a schematic perspective view of a pain management system in accordance with one embodiment, including compression shorts that have moisture wicking technology and a moisture barrier and a pouch configured to fit a cold therapy pack.

FIG. 2 is a top view of the cold therapy pack of the pain management system illustrated in FIG. 1;

FIG. 3 is a bottom view of the cold therapy pack of the pain management system illustrated in FIGS. 1 and 2;

FIG. 4 is a bottom view of the pain management system illustrated in FIG. 1 with portions cut away;

FIG. 5 is a rear elevation view of the pain management system illustrated in FIGS. 1, 3, and 4;

FIG. 6 is a bottom schematic view of the pain management system illustrated in FIGS. 1 and 3-5, with portions cut away; and

FIG. 7 is a schematic of a dressing gauze pad that may be included in an embodiment of the pain management system shown in FIGS. 1 and 3-6.

### DETAILED DESCRIPTION

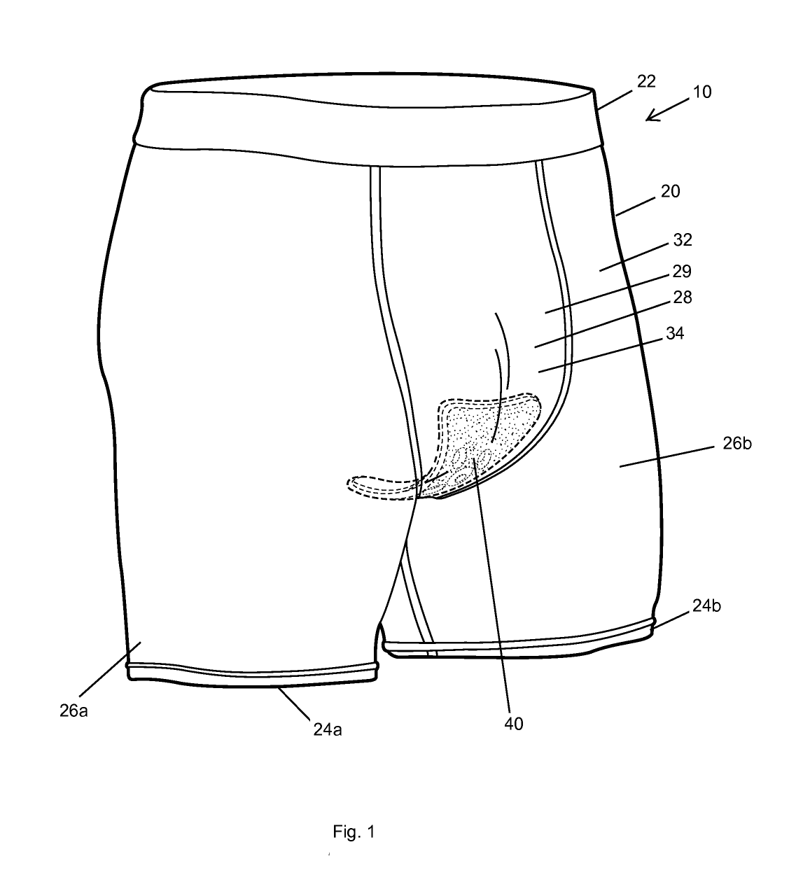

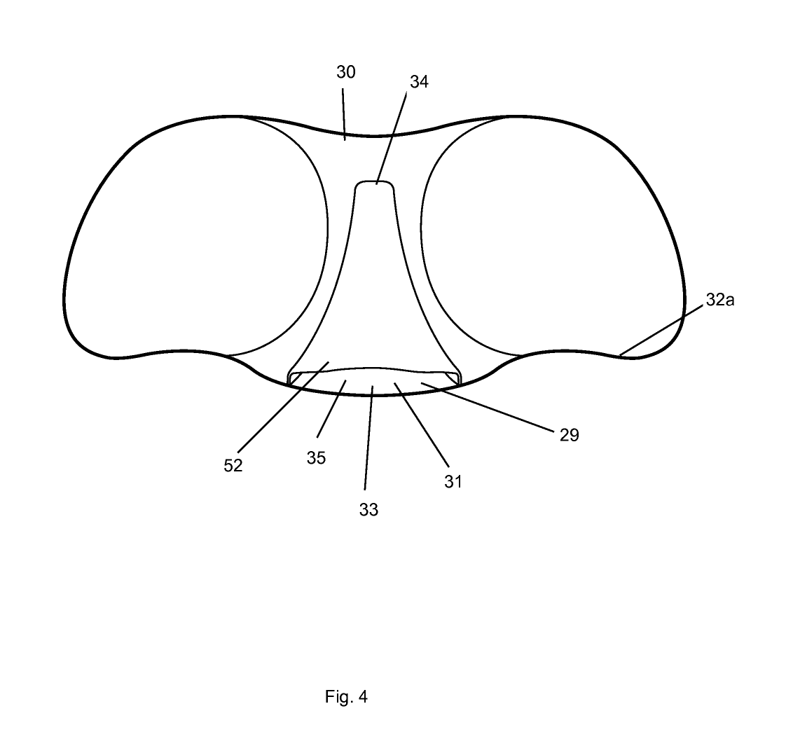

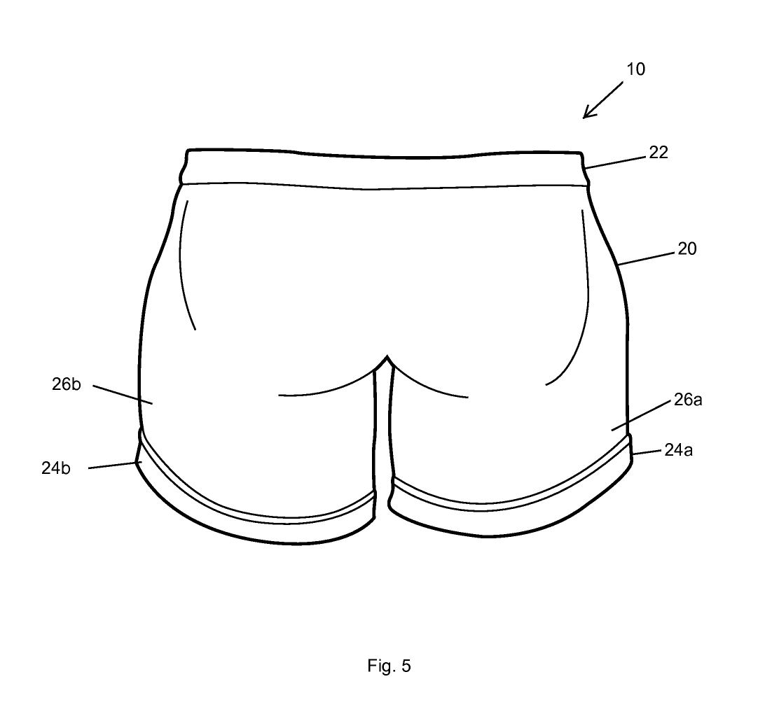

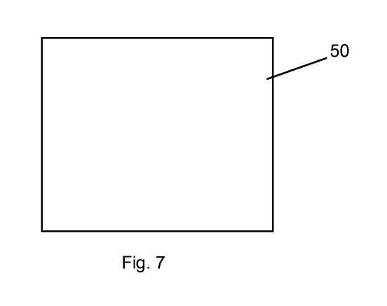

Referring to FIGS. 1-6 generally, the pain management system 10 in the embodiment shown includes a support undergarment 20 and a therapy pack 40. In the exemplary embodiment, the support undergarment 20 may be boxer-brief style underwear or shorts 20. The pain management system may also include dressing 50 such as the dressing 50 shown in FIG. 7.

In the embodiment shown, the shorts 20 can be configured to be form-fitting for a patient wearing the shorts 20. The shorts can include a waistband 22 at an opening at the anterior end and thigh bands 24a,b at two openings on the posterior end opposing the anterior end. The shorts 20 can have leg portions 26a,b on the opposing dorsal and ventral sides, respectively. A groin portion 28 can be located between the two leg portions 26,b. The shorts can also have a rear portion 30 that opposes the groin portion 28. The shorts 20 can also include a base layer 32 and a secondary layer that forms a pouch 34. The leg portions 26a,b, groin portion 28, and rear portion 30 are all comprised of the base layer 32. The base layer 32 also has an inner or proximal side 32a configured to touch a patient's skin during use, and an outer or distal side 32b opposing the proximal side 32a. The groin portion 28 also includes the secondary layer that forms the pouch 34.

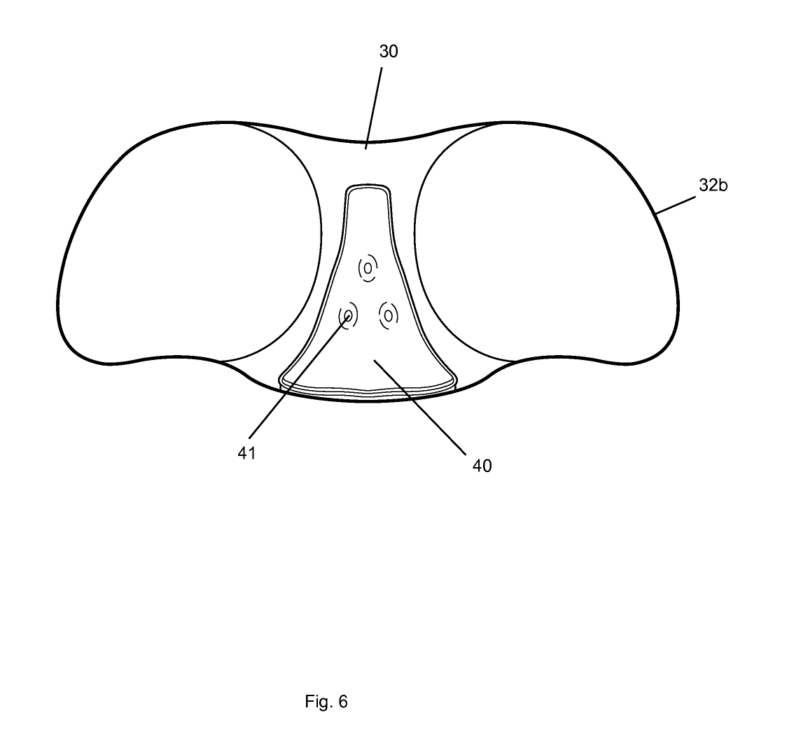

In the embodiment shown in the figures, the pouch 34 extends from the groin portion 28 to the rear portion 30 of the shorts 20. The pouch 34 can be configured to removably secure the therapy pack 40 between the distal side 32b of the base layer 32 and the secondary layer that forms the pouch 34. Specifically, the pouch 34 and the base layer can form an opening 35 through which the therapy pack 40 can be placed in and removed from the pouch 34. As shown in FIGS. 1 and 6, when worn by the patient, the pouch is configured so that the therapy pack 40 fits over the patient's groin region in such a way that the patient's mobility does not compromise the placement of the therapy pack 40. In alternative embodiments, the pain management system may further include a method of securing the opening 35 of the pouch 34.

The shorts 20 can have compression properties that provide support to the patient when worn. Accordingly, in one embodiment, the shorts 20 can be made from a polyester/spandex blend of materials or any material blend that would provide similar compression features. In an alternative embodiment, the compression shorts can be made from multiple types of materials and material blends to provide varying levels of compression for different anatomical areas of a patient. For example, the compression level for the groin portion 28 of the base layer 32 can be higher than the compression level for the waist band 22 and thigh bands 24a,b such that the groin portion 28 is a support panel 29. Additionally, in another alternative embodiment, the compression shorts may be configured for the positioning of a therapy pack in a different region or a different part of the groin region.

The shorts 20 can also entirely or partially consist of an antimicrobial material 31. For example, the compression shorts 20 can be treated with an antimicrobial spray to form an antimicrobial material 31. This spray may last through 7 days of wear. In one embodiment, shorts 20 may be treated with Fabricaide fabric treatment available from Coating Specialists, LLC (http://www.antimicrobial.com/products/fabricaide/). In another embodiment, shorts 20 may be treated with AX AntiBacterial Fabric Treatment available from Lifesystems (http://www.lifesystems.co.uk/psec/accessories/ax_antibacterial_fabric.htm). Alternatively, only a portion of the shorts 20 can have a spray treatment to form an antimicrobial material 31. In one embodiment, the proximal side 32a in the groin portion 28 of the base layer 32 of the shorts 20 that contact the patient's groin area can be treated with such a spray. In yet another embodiment, the entire groin portion 28 of the base layer 32 can be formed of material treated with antimicrobial spray. The shorts can also be entirely or partially formed from material that has been treated in other ways so that it has antimicrobial properties. For example, the material can be dipped in an antimicrobial coating to form an antimicrobial material 31. In another embodiment, silver thread can be woven in to material of the shorts 20 to form an antimicrobial material 31.

The shorts 20 can be entirely or partially made of material that is moisture wicking. Such material exhibits properties that, when the shorts 20 are worn by the patient, the material pulls moisture away from the patient's skin to the surface of the fabric so that the moisture can evaporate. For example, such material can transfer moisture from a patient's skin by capillary action to the outer surface of the material where it will evaporate. Synthetic materials which exhibit these properties, such as polyester or microfiber-based fabrics, can be used. In one embodiment, the pouch 34 is made of moisture wicking material 52. In another embodiment, the entire base layer 32 can be made of moisture wicking material.

In yet another embodiment, the shorts 20 can be entirely or partially made of material that is a moisture barrier. Such material exhibits properties that, when the shorts 20 are worn by the patient, such material blocks moisture from touching the patient's skin. For example, in the embodiment shown, the distal layer 32b in the groin portion 28 of the base layer 32 of the shorts 20 can include a moisture barrier 33 that blocks moisture from the therapy pack 40 located in pouch 34 from the patient's skin.

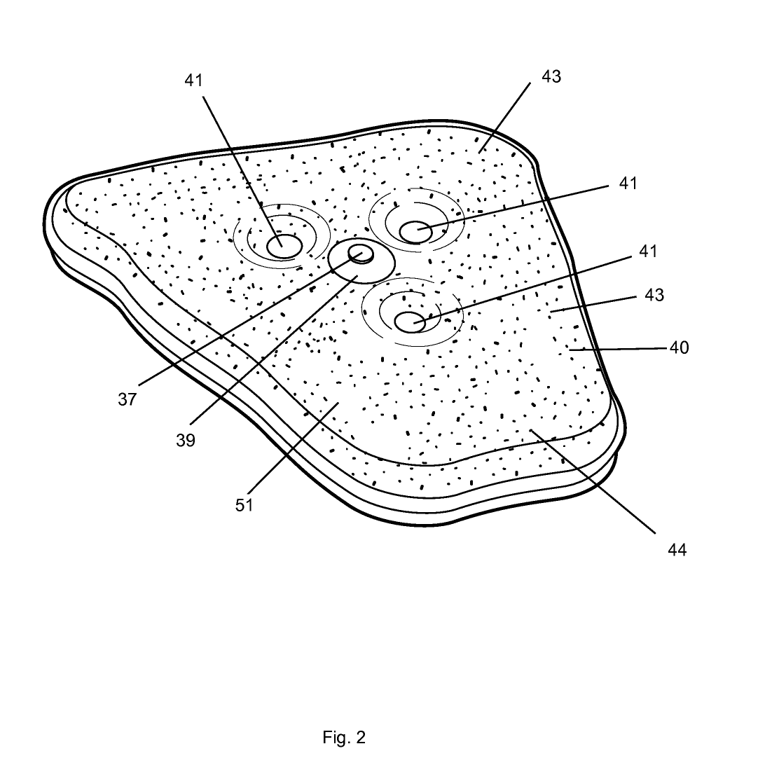

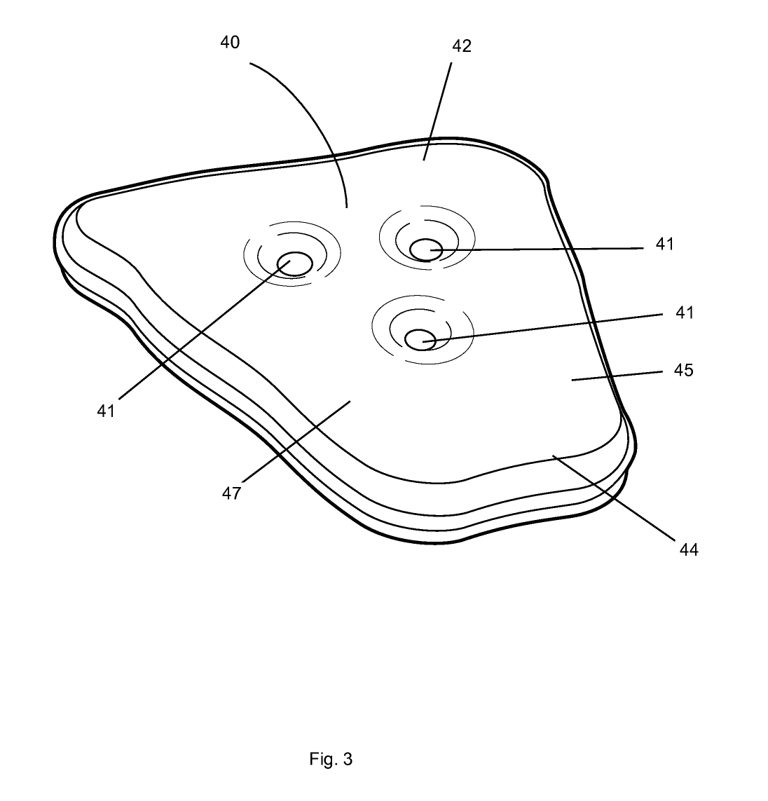

FIGS. 2 and 3 show the therapy pack 40. In the embodiment shown, the pack 40 has a generally trapezoidal shape, including a distal region 42 and a proximal region 44, and is configured to fit within the pouch 34 of the shorts 20. As shown in FIG. 1, the compress is moldable and can be configured to fit around the patient's groin region to form a contoured design. The therapy pack 40 can be a cold therapy pack configured to absorb heat. In one embodiment, the therapy pack 40 is formed from a multi-layer material that includes an inner polyethylene layer and contains a plurality of discrete, non-water soluble, hydrophilic absorbers hydrated in a liquid that consists primarily of water. In this embodiment, the absorbers can be shaped as beads. The compress can further include an air permeable membrane comprised of a microporous membrane such as poly-tetra-fluoro-ethylene (PTFE) or an acrylic provided by Metis Products, LLC of San Diego, Calif. U.S. Pat. No. 5,800,491, entitled “Thermal Therapy Devices and Methods of Making the Same” is directed at a process for making a type of therapy pack 40 and is hereby incorporated in its entirety herein. In this embodiment, a heat absorbing material 51 in the therapy pack 40 is designed to allow for long lasting pain treatment that also minimizes the risk of frostbite or cold burns. This risk is minimized because the material in the pack adjacent to the skin quickly melts. The frozen material in the remainder of the pack is then largely spaced away from direct contact with the skin. These properties are especially important for applications involving sensitive areas of skin, such as male genitals.

FIG. 2 shows the top side 43 of the therapy pack 40. The therapy pack includes a hole 37 for filling the pack with heat absorbing material 51. A plug 39 cover the hole 37 once the pack 40 is filled with the heat absorbing material 51. In some embodiments, the heat absorbing material in the therapy pack 40 can include a cold therapy gel 51 that absorbs heat. In another embodiment, the therapy pack 40 may include cold therapy beads 51. The therapy pack 40 may also include one or more dimples 41. Dimples 41 connect the bottom side 43 to a top side 45. In some embodiments, dimples 41 facilitate the distribution of heat absorbing material 51 in the therapy pack 40.

FIG. 3 shows the bottom side 45 of the therapy pack 40. Dimples 41 are also shown on the top side 45. In some embodiments, the therapy pack 40 may have a thickness of approximately 0.25-0.5 inches (6.35-12.7 mm) thick. The thickness of the therapy pack may be increased to provide a longer cooling time. In one embodiment, the top side 45 of the therapy pack 45 may further include a fabric layer 47. The fabric layer 47 may be used to secure the therapy pack 40 in the pouch 34 by increasing the amount of friction between the therapy pack 40 and the pouch 34. The fabric layer 47 may also absorb condensation to minimize condensation. The fabric layer 47 may also have an anti-microbial treatment.

The pain management system 10 can further include dressing 50 shown in FIG. 7. For example, the dressing can be in the form of sterile gauze pads that are approximately 1 Inch×1 inch in size. For example, SilverPro by BREG or Select Silver by Milliken Medicalmay be used. The dressing is configured to be placed on the patient's incision site following a surgical procedure. The dressing may be applied by doctor or patient for 24 hours or until bleeding stops and may be changed as needed.

The pain management system 10 described above can be sold as a compilation of components or as individual components. If sold as a group of components, such a product can include the shorts 10 and two therapy packs 40. The product can also include two 1 inch×1 inch squares of dressing gauze pads.

The embodiments described in connection with the illustrated embodiments have been presented by way of illustration, and the present invention is therefore not intended to be limited to the disclosed embodiments. Furthermore, the structure and features of each the embodiments described above can be applied to the other embodiments described herein, unless otherwise indicated. Accordingly, those skilled in the art will realize that the invention is intended to encompass all modifications and alternative arrangements included within the spirit and scope of the invention, for instance as set forth by the appended claims.

What is claimed is:

1. A pain management system comprising:
a support undergarment with a pouch configured to removeably secure a therapy pack, wherein the support undergarment comprises an anti-microbial material and at least one moisture barrier; and
a therapy pack, wherein the therapy pack is configured to form into a contoured design.

2. The pain management system of claim 1 wherein the support undergarment further comprises support panels configured to provide stability to male genitals.

3. The pain management system of claim 1 wherein the support undergarment is boxer-brief style underwear.

4. The pain management system of claim 1 wherein the therapy pack comprises cold therapy gel.

5. The pain management system of claim 1 wherein the therapy pack comprises cold therapy beads.

6. The pain management system of claim 5 wherein the therapy pack comprises material configured to minimize the risk of frostbite when applied to male genitals.

7. The pain management system of claim 1 wherein the pain management system further comprises dressing gauze pads.

8. The pain management system of claim 1 wherein the support undergarment has an outer side and an inner side, and wherein the therapy pack is removably secured to the outer side, and wherein the at least one moisture barrier is configured to minimize transfer of moisture from the therapy pack to an inner side of the support undergarment.

9. The pain management system of claim 1 wherein the support undergarment further comprises moisture wicking material.

10. The pain management system of claim 9 wherein the support undergarment comprises a base layer that further comprises a groin portion with an inner side and an opposing outer side, and wherein the base layer comprises the moisture wicking material and the inner side of the groin portion at least partially comprises the anti-microbial material and the outer side of the groin portion at least partially comprises the at least one moisture barrier.

11. The pain management system of claim 1 wherein the contoured design of the therapy pack is contoured to fit around male genitals.

12. A kit comprising:
a support undergarment with a pouch configured to removably secure a therapy pack, wherein the support undergarment comprises and anti-microbial material and at least one moisture barrier;
a plurality of therapy packs, wherein each therapy pack is configured to form into a contoured design; and
a plurality of dressing gauze pads.

13. A method for managing pain comprising:
compressing an area of a human body,
applying a therapy pack to the area,
providing antimicrobial treatment to the area, and
minimizing moisture on the area.

14. The method of claim 13 wherein the step of compressing further comprises using a support undergarment.

15. The method of claim 13 further comprising a step of cooling the area.
